(19) United States
(12) Patent Application Publication
(10) Pub. No.: US 2005/0171547 A1
(43) Pub. Date: Aug. 4, 2005

(54) Surgical instrument, and related methods

(76) Inventor: Tony N. Aram, Fairfax, VA (US)

Correspondence Address: David Taylor;Liniak, Berenato & White, Suite 240, 6550 Rock Spring Drive, Bethesda, MD, 20817, US

(21) Appl. No.: 10/765,950

(22) Filed: Jan. 29, 2004

(51) Int. Cl. A61B 17/58

(52) U.S. Cl. 606/74

(57) ABSTRACT

A surgical instrument is provided, as is a method for stabilizing and facilitating recovery of injured bone within a living body. The surgical instrument includes a flexible cable and a plurality of permanent bone-contacting enlargements fixedly attached to the flexible cable. The bone-contacting enlargements are spaced apart from one another to provide linking cable portions alternating with the spaced bone-contacting enlargements. The spaced relationship between the bone and the linking cable portions provide channels along the bone length for permitting vascular circulation across a region of the injured bone to which the surgical instrument is applied.

### BACKGROUND OF THE INVENTION

1. Field of the Invention

This invention generally relates to the field of surgical instruments, and possesses particular applicability to the field of cerclage instruments and other mending devices and methods for repairing injured (e.g., broken or fractured) bones or reconstructing bones.

2. Description of the Related Art

Orthopedic surgery comprises, among other things, the mending of bone fractures, and the reconstruction of bones, including, for example, reconstructive hip, knee, shoulder, and elbow replacements. In orthopedic surgery, it is common to implant a permanent cerclage into a living body to secure a bone, bones, or bone fragments. Cerclages generally encircle or loop around the bone(s) or bone fragments, and are tightened to hold the bone(s) or bone fragments together. The tight fit of the cerclage facilitates bone healing and inhibits crack formation and/or propagation in the bone.

Surgical cables have become perhaps the most widely accepted and trusted cerclage amongst orthopedic surgeons. The wide acceptance of surgical cables in the orthopedic field is believed to be due to several factors. Surgical cables possess physical properties well matched for their intended function of achieving stabilization and promoting recovery of an injured (e.g., broken, fractured, or reconstructed) bone(s). Surgical cables also have a combination of flexibility and longitudinal stiffness that facilitates looping of the cables around injured bones. Additionally, orthopedic surgeons have generally become accustomed and comfortable with modern cable tensioning and clamping devices, many of which are designed specifically for use with conventional cables. Examples of cable-tensioning and cable-clamping devices are found in U.S. Pat. No. 6,595,994 and U.S. Pat. No. 5,415,658, respectively.

However, the constrictive fit of cerclages such as cables around the bone have been shown to inhibit the vascular circulation in the bone across the bone area fitted with the cerclages, and can lead to necrosis and non-healing. These problems may require a second operation, removal of cerclages, and bone grafting, which inconveniences the patient and presents an inherent risk of complications.

Various efforts have been made to design cerclages that counteract or avoid the problems associated with necrosis. For example, U.S. Pat. No. 4,263,904 discloses osteosynthesis device comprising a circular bracelet having three inwardly directed, pointed bosses pressed into the bone. A cerclage comprising a fabric strip with transverse ribs is disclosed in U.S. Pat. No. 4,667,662. In U.S. Pat. No. 5,127,413, a flexible sinuous suture comprising resilient monofilament material is disclosed. A drawback common to each of these devices is their incompatibility with accepted cable-tensioning and cable-clamping equipment. Many orthopedic surgeons have become accustomed to and reliant upon surgical cables and surgical cable tensioning and clamping equipment. Consequently, many orthopedic surgeons are resistant to significant changes in the equipment they use.

### OBJECTS OF THE INVENTION

It is an object of the invention to provide a surgical instrument that is compatible with conventional cable-tensioning and/or cable-clamping equipment.

It is a further object of the invention to provide a surgical instrument that avoids or circumvents problems associated with vascular circulation inhibition seemingly inherent to surgical cables.

It is yet another object of this invention to provide methods for making and using the surgical instrument of the present invention to repair, stabilize, or otherwise mend an injured bone, such as an injured or reconstructed bone, of a living being.

### SUMMARY OF THE INVENTION

To achieve one or more of the foregoing objects, and in accordance with the purposes of the invention as embodied and broadly described in this document, according to a first aspect of this invention there is provided a surgical instrument for stabilizing and facilitating recovery of injured (e.g., broken, fracture, or reconstructed) bone within a living body. The surgical instrument comprises a flexible cable having a first end, a second end, and a length between the first and second ends sufficient to wrap around the injured bone. The surgical instrument further comprises a plurality of permanent bone-contacting enlargements fixedly attached to the flexible cable between the first and second ends. The bone-contacting enlargements are spaced apart from one another to providing linking cable portions alternating with the spaced bone-contacting enlargements.

A second aspect of this invention provides a method for stabilizing and facilitating recovery an injured bone within a living body. The method comprises providing a surgical instrument comprising a flexible cable having first and second ends and a length, and a plurality of permanent bone-contacting enlargements fixedly attached to the flexible cable between the first and second ends and spaced apart from one another to provide linking cable portions alternating with the spaced bone-contacting enlargements. The surgical instrument is passed about the injured bone to contact the bone-contacting enlargements and the injured bone with one another. The bone-contacting enlargements position the linking cable portions in spaced relationship to the injured bone. The flexible cable is tensioned about a constricted region of the injured bone while the bone-contacting enlargements retain the linking cable portions in spaced relationship to the injured bone for permitting vascular circulation in the bone across the constricted region of the bone. The surgical instrument is then secured about the constricted area of the injured bone.

### BRIEF DESCRIPTION OF THE DRAWINGS

The accompanying drawings are incorporated in and constitute a part of the specification. The drawings, together with the general description given above and the detailed description of the preferred embodiments and methods given below, serve to explain the principles of the invention. In such drawings:

FIG. 1 is a partial schematic view of an embodiment of the surgical instrument of the present invention, depicting the surgical instrument being looped around an injured bone with the assistance of a cable passer;

FIG. 2 is a cross section of the surgical instrument of FIG. 1 looped around the injured bone (shown in part), depicting the surgical instrument secured, in part, with a conventional connecting device;

FIG. 3 is a partial schematic view of the surgical instrument of FIG. 1 looped the injured bone (shown in cross section), depicting the cable-connecting device and a cable tensioning device for securing and tightening the surgical instrument;

FIG. 4 is a cross section of the injured bone, depicting the surgical instrument looped, tensioned, and secured about the bone;

FIG. 5 is a partial schematic view depicting multiple surgical instruments identical to FIG. 1 separately looped, tensioned, and secured about the injured bone;

FIG. 6 is a cross section of a modified clamping device used with an embodiment of the method of the present invention;

FIG. 7 is a cross sectional of another modified clamping device used with an embodiment of the method of the present invention;

FIG. 8 is a partial schematic view of the surgical instrument of FIG. 1, depicting the surgical instrument passed around an injured bone containing a hip prosthesis a plurality of times;

FIG. 9 a partial schematic view of the surgical instrument of FIG. 1, depicting the surgical instrument used in combination with a surgical plate; and

FIG. 10 is a sectional view of a conventional clamping device.

### DETAILED DESCRIPTION OF CERTAIN PREFERRED EMBODIMENTS AND METHODS OF THE INVENTION

Reference will now be made in detail to the presently preferred embodiments and methods of the invention as illustrated in the accompanying drawings, in which like reference characters designate like or corresponding parts throughout the drawings. It should be noted, however, that the invention in its broader aspects is not limited to the specific details, representative devices and methods, and illustrative examples shown and described in this section in connection with the preferred embodiments and methods. The invention according to its various aspects is particularly pointed out and distinctly claimed in the attached claims read in view of this specification, and appropriate equivalents.

It is to be noted that, as used in the specification and the appended claims, the singular forms “a,” “an,” and “the” include plural referents unless the context clearly dictates otherwise.

According to an embodiment of the invention, a surgical instrument is provided for stabilizing and facilitating recovery of injured bone within a living body. The surgical instrument is intended for both human applications and veterinary applications. Examples of bone injuries for which the surgical instrument of the invention may be applied includes broken or fractured bones (e.g., femur, tibia, humerus, patella, etc.), prophylactic banding of the femur during press fit total hip replacement, stabilization of cortical on lay strut grafts, trochanteric reattachments, and in the fixation of flat bones such as the sternum after open chest surgery.

The surgical instrument of an embodiment of the invention comprises a flexible cable having a first end, a second end, and a length between the first and second ends sufficient to wrap around the injured bone. The surgical instrument further comprises a plurality of permanent bone-contacting enlargements fixedly attached to the flexible cable between the first and second ends. The bone-contacting enlargements are spaced apart from one another to provide linking cable portions alternating with the spaced bone-contacting enlargements.

Referring more particularly to the figures, a surgical instrument is illustrated and generally designated by reference numeral 10. The surgical instrument comprises a flexible cable 12 having sufficient length and flexibility to permit the cable 12 to be wrapped around the circumference of a bone, such as the humerus or femur. Although flexible radially, the cable 12 is preferably axially inelastic, i.e., substantially incapable of longitudinal stretching. Representative materials of which the cable may be made include metals and metal alloys, such as stainless steel or cobalt chrome. The cable 12 may be multi-strand or monofilament, depending upon the intended use of the instrument 10. (Monofilament cables 12 are more typical for veterinary applications, due to the lighter weight of the patient and the low cost.) A non-exhaustive list of cable suppliers comprises Howmedica/Stryker, which produces 1.6 mm and 2.0 mm DALL-MILES cables; Acumed, which produces 1.6 mm and 2.0 mm OSTEO-CLAGE cables; and Zimmer/Pioneer, which produces 1.3 mm and 1.8 mm CABLE-READY SYSTEM cables.

The surgical instrument 10 further comprises a plurality of bone-contacting enlargements 14. In the illustrated embodiment, the bone-contacting enlargements 14 comprise beads having a substantially spherical periphery. The bone-contacting 14 may undertake other shapes and configurations, but are preferably obtuse, i.e., blunt and unpointed. For example, the bone-contacting enlargements of an alternative embodiment comprise annular ribs having rounded peripheries or polygonal shaped (e.g., pentagonal to octagonal) peripheries.

The bone-contacting enlargements 14 are preferably made of a permanent material. As used herein, permanent means that the material resists resorption or is substantially non-resorbable into the living being's body during the expected natural life span of the living being. It is currently envisioned that the bone-contacting enlargements 14 comprise polymeric material, and preferably a high molecular polymeric materials, such as a polyolefin such as polyethylene. The polymeric bone-contacting enlargements may be fixedly attached to the cable 14 using, for example, compression molding techniques. The bone-contacting enlargements 14 alternatively comprise a metal or metal alloy, such as stainless steel or cobalt chrome. Metal enlargements may be fixed to a cable 12 by boring a diametric hole through the enlargements, passing the cable 12 therethrough, then compressing the enlargements onto the cable 12 with, for example, a hydraulic press.

The bone-contacting enlargements 14 are spaced apart from one other along the length of the flexible cable 12 to define linking cable portions 16 of the flexible cable 12 extending between the bone-contacting enlargements 14. In the illustrated embodiments, the bone-contacting enlargements 14 are greater in dimension than the lesser diameter, adjacent linking cable portions 16. More preferably, the bone-contacting enlargements 14 are circumferentially non-directional, i.e., circumferentially surround the flexible cable 12 (for all 360 degrees of the cable 12 periphery). The non-directional bone-contacting enlargements 14 are preferred because their contact with an injured bone 20 is not lost or otherwise adversely affected by accidental twisting of the cable 12, for example, as might occur when the surgical instrument 10 is passed around the bone 20.

The linking cable portions 16 and the bone-contacting enlargements 14 alternate in sequence with one another. This alternating arrangement may encompass a set or sets of two or more bone-contacting enlargements 14 immediately adjacent and contacting one another, with the linking cable portions 16 alternating with the sets of enlargements 14. The bone-contacting enlargements 14 preferably all have the same axial length, although it should be understood that the bone-contacting enlargements 14 may have non-uniform axial lengths, i.e., different axial lengths from one another. Likewise, the linking cable portions 16 preferably all have the same axial length, although it should be understood that the linking cable portions 16 may have non-uniform axial lengths, i.e., different axial lengths from one another. For example, the bone-contacting enlargements 14 of embodiments of the invention have a diameter of, for example about 4 mm (e.g., for 20 mm diameter bones) to about 6 mm (e.g., for 40 mm diameter bones) for multi-strand cable. Enlargements fixed on monofilament cables generally may have a slightly lesser diameter. The axial lengths of the linking cable portions 16 of the illustrated embodiment preferably are selected to provide a ratio of 2.8 for the distance between adjacent enlargement centers to the enlargement diameter. For example, adjacent enlargements having diameters of 4 mm will be spaced about 7 mm apart (so that the distance between adjacent enlargement centers would be 11 mm, which divided by the 4 mm enlargement diameter gives a ratio of about 2.8 (actually 2.75)). Thus, the linking cable portions 16 preferably yet optionally have respective axial lengths greater in dimension than the bone-contacting enlargements 14.

Preferably, at least one end portion 18 of the cable 12 is free of bone-contacting enlargements 14. The end portion 18 of surgical instrument 10 that is free of the bone-contacting enlargements is also referred to herein as “enlargement-free end portion 18”. The enlargement-free end portion 18 is preferably sufficient in length to facilitate compatibility of the surgical instrument 10 with conventional cable-tensioning and clamping devices, as discussed in greater detail below. For purposes of convenience and explanation, in this detailed explanation the terms “proximal” and “proximal direction” shall mean closer to or towards the enlargement free-end portion 18 that engages the cable-tensioning device, and the terms “distal” and “distal direction” shall mean farther away from the enlargement-free end portion 18 that engages the cable-tensioning device. (Optionally, both end portions of the cable 12 may be free of bone-contacting enlargements 14. This optional embodiment is particularly useful with certain cable-tensioning and/or clamping systems, e.g., the Zimmer system and others requiring that both end portions pass through a crimp to engage the tensioner.)

A method for stabilizing and facilitating recovery of injured bone within a living body will now be discussed in detail. It is to be understood that the following method is not exhaustive of the methods in which the surgical instrument of this invention may be used.

In accordance with embodiments of the invention, a method is provided for stabilizing and facilitating recovery of injured bone within a living body. The method comprises providing a surgical instrument comprising a flexible cable and a plurality of permanent bone-contacting enlargements. The flexible cable has first and second ends and a length between the ends sufficient to wrap around the injured bone. The bone-contacting enlargements are fixedly attached to the flexible cable between the first and second ends and are spaced apart from one another to provide linking cable portions alternating with the spaced bone-contacting enlargements. The surgical instrument is passed about a constricted region of the injured bone to contact the bone-contacting enlargements with the injured bone, and the linking cable portions are positioned in spaced relationship to the injured bone. The flexible cable is tightened about a constricted region of the injured bone while the bone-contacting enlargements retain the linking cable portions in spaced relationship to the injured bone for permitting vascular circulation across the constricted region. The surgical instrument is secured about the injured bone to facilitate bone recovery and prevent aggravation of the injury.

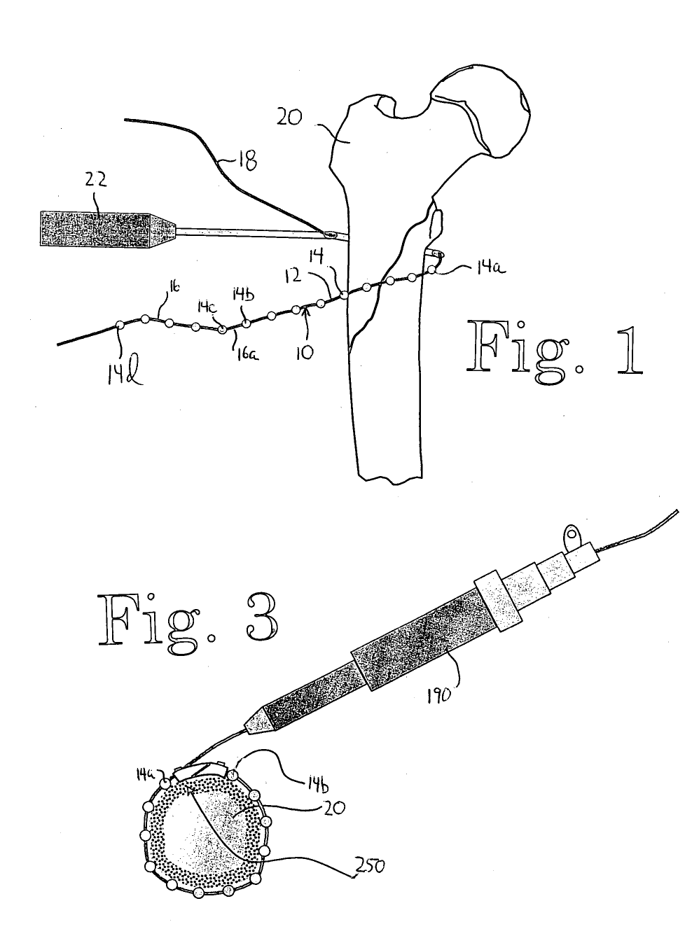

FIG. 1 is a partial schematic view of a surgical procedure step showing the surgical instrument 10 being passed around an injured area of injured bone 20 (shown without the other tissues of the patient, for purposes of convenience). In the illustrated embodiment a cable passer 22 is used to guide the surgical instrument 10 behind and around the injured bone 20 from the incision area (not shown). Due to concerns comprising compatibility of the surgical instrument 10 with existing cable passers 22 and other existing devices, such as cable tensioners, as described below, it is preferred that the enlargement-free end portion 18 be passed retrograde around the injured bone 20. As can be seen from FIG. 1, a known cable passer 22 may be used with the surgical instrument 10 of embodiments of the invention. After the enlargement-free end portion 18 has been passed around the bone 20, the cable passer 22 may be disposed of, and the cable 12 may be pulled by hand or with a tool.

As shown in FIG. 3, preferably the enlargement-free end portion 18 of the surgical instrument 10 is continually fed and passed around the injured bone 20 until a first bone-contacting enlargement 14a (FIG. 3) reemerges from behind the bone 20 so that the bone-contacting enlargements 14 encircle the injured bone 20. The first bone-contacting enlargement 14a preferably but not necessarily will form part of the cerclage that will contact the bone 20 and remain within the body.

The enlargement-free end portion 18 is then fed into a clamp, crimp, connector, or other equivalent or suitable securing device. An example of a connecting device that may be used with the present invention is disclosed in U.S. Pat. No. 5,415,658, the complete disclosure of which is incorporated herein by reference. This connecting device is reproduced in FIG. 10 herein and is briefly described herein. It is to be understood that the referenced connecting device is merely illustrative, and not exhaustive of the connecting devices and other clamping and securing devices that may be used with the surgical instrument and methods of the invention.

The known connecting device 110 shown in FIG. 10 comprises a body 114 having projections 124a in contact with an injured bone 20. The body 114 comprises a first cable receiving bore 128 extending from an end 116 to an open aperture 132, and a second cable receiving bore 130 extending from an end 118 to the open aperture 132. A concave inward side 120 extends between the ends 116, 118 and faces the bone 20. The bores 128, 130 have axes that occupy a common plane and are angled, for example, 110 to 160 degrees relative to one another. The first cable-receiving bore 128 includes an annular step 138 between inner portions 134 and 136. The end of a cable 112 occupying first cable-receiving bore 128 carries a metal enlarged tip 140 swaged to the end of the cable 112 to fit into the bore portion 134, but sized not to pass through the bore portion 136. The end portion 142 of the cable 112 passing through second cable-receiving bore 130 is fed through aperture 132 and pulled in a tensioning device (described below) to provide the desired tension to the loop defined by the cable 112. Threaded screw 144 is advanced (e.g., via a screw driver fitting into driving aperture 146) through hole 145 to compress the cable 112 between the screw 114 and sleeve 150 to provide a compressive, frictional retention of the cable 112. As the screw 114 is advanced, flat face 154 enters into engagement with annular seat 156 to terminate screw advancement. The end portion 142 of the cable 112 may then be cut, for example, at 158.

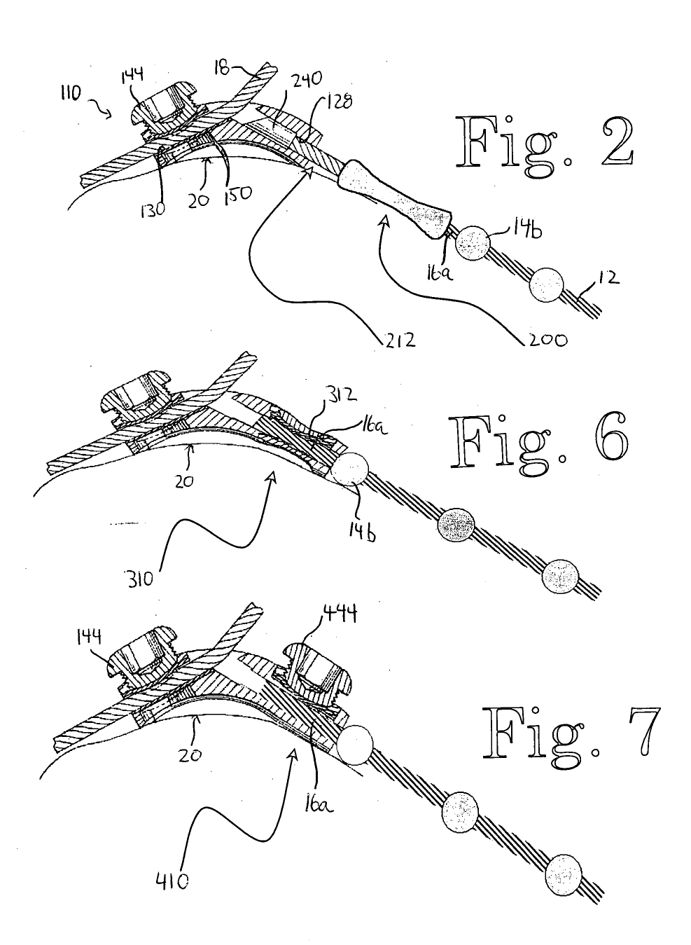

An embodiment in which the connecting device 110 of FIG. 10 is used without modification with an embodiment of the surgical cable of the present invention will now be described with reference to FIGS. 2 and 3. After the enlargement-free end portion 18 of the surgical instrument 10 is passed around the bone 20 to place the bone-contacting enlargements 14 in contact with the bone 20 as described above, the enlargement-free end portion 18 is fed through bore 130 of the connecting device 110. However, absent modification to the connecting device 110, the bone-contacting enlargements 14 of the illustrated embodiment are too large to fit through bore 128 of the connecting device 110. Accordingly, the cable 12 of the surgical instrument 10 is cut with a known cable cutter or pliers between two adjacent bone-contacting enlargements 14b and 14c, preferably at a position closer to, if not immediately against, the more distal bone-contacting enlargement 14c, leaving a linking-cable-portion free end 16a for insertion into a conventional crimp 200 (FIG. 2). The selected bone-contacting enlargement 14b preferably will form part of the cerclage that will contact the bone 20 and remain within the body, and more preferably will be adjacent to enlargement 14a (with connecting device 110 interposed between enlargements 14a and 14b).

The connecting device 110 is provided with a truncated cable portion 212 having an end with a cable tip enlargement 240 swaged thereon. The cable tip enlargement 240 is sized to fit into the bore portion 134 (see FIG. 10), but to prevent passage through the bore portion 136 (FIG. 10) of the connecting device 110. The opposite end of the truncated cable portion 212 is placed into the crimp 200, which is then pinched with a conventional crimping device (not shown) to link the truncated cable portion 212 to the free end 16a of the cable 12.

As discussed above, the enlargement-free end portion 18 is fed into a cable tensioning device 190, shown in FIG. 3. An example of a tensioning device that may be used with the present invention is disclosed in U.S. Pat. No. 6,595,994, the complete disclosure of which is incorporated herein by reference. It is to be understood that the referenced tensioning device is merely illustrative, and not exhaustive of the tensioning devices that may be used with the surgical instrument and methods of the invention.

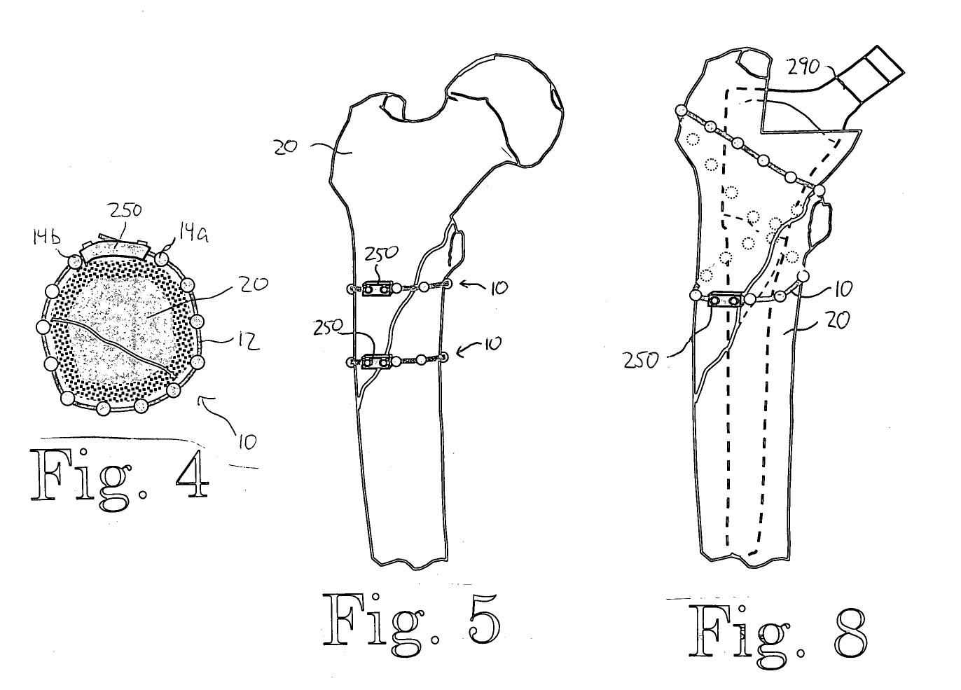

The referenced tensioning device of the '994 patent comprises an annular body having a tubular shaft for receiving the enlargement-free end portion 18 therethrough. The tensioning device is provided with locking and tensioning mechanisms for securing the end portion 18 and tensioning the surgical instrument 10 around the injured bone. After the surgical instrument 10 has been placed under tension, the threaded screw 144 of the connecting device 110 is advanced through hole 145 (FIG. 10) to compress the cable end portion 18 between the screw 114 and sleeve 150 to provide a compressive, frictional retention of the surgical instrument 10. The unused portion of the enlargement-free end portion 18 may then be cut and removed, leaving the surgical instrument 10 looped around the injured bone 20, as shown in FIG. 4. This procedure may be repeated multiple times along the length of a single fracture, as shown in FIG. 5. For simplification and convenience purposes, and to stress the compatibility of the surgical instrument with other connecting devices, the connecting device 110 and the crimp 200 are illustrated collectively as a package 250 in FIGS. 3, 4, 5, and 8.

The above-embodied method presupposes that the length of the enlargement-containing portion of the cable 12, i.e., the length between enlargement 14a and the most distal enlargement 14d, is sufficiently greater than the circumference of the injured bone 20, so that one or more of the enlargements 14 are unused, i.e., do not form part of the cerclage that will contact the bone 20 and remain within the body. In these embodiments, the unused length of the enlargement-containing portion of the cable 12 is removed, e.g., by cutting a linking cable portion 16a between 14b and 14c in the above embodiment. It is within the scope of this invention to provide a different surgical instruments having different enlargement-containing portion lengths from one another, and to pre-select a given one of the surgical instruments 10 having an enlargement-containing portion length that will permit the surgical instrument to be passed around the bone 20 once (or multiple times) without leaving residual, unused enlargements 14 to be removed via cutting. This pre-selection process likely will involve a certain degree of estimation on the part of the orthopedic surgeon, and possibly may complicate the surgery if an incorrect length surgical instrument 10 is pre-selected.

Another embodiment of a connecting device useful in the method of an embodiment of the present invention is illustrated in FIG. 6. In this embodiment, the crimp 200 and connecting device 110 of FIG. 2 have been integrated to provide a modified connecting device 310 having a crimpable body portion 312. After passing the surgical instrument 10 around the injured bone 20 and cutting the cable 12 to provide the linking cable portion free end 16a (as discussed above), the free end 16a is pinched within the crimpable body portion 302. Advantageously, the provision of the crimpable body portion 302 circumvents the use of separate truncated cable portions, e.g., 212 in FIG. 2.

Yet another embodiment of a connecting device useful in the method of an embodiment of the present invention is illustrated in FIG. 7. The connecting device 410 includes a second threaded screw 444 provided in lieu of the crimpable body portion 312 of FIG. 6. The second threaded screw 444 may be constructed and operated in much the same manner as the first threaded screw 144. After passing the surgical instrument 10 around the injured bone 20 and cutting the cable 12 to provide the linking cable portion free end 16a (as discussed above), the second threaded screw 444 is advanced to compressively retain the free end 16a of the surgical instrument 10. Advantageously, the provision of the second threaded screw 444 circumvents the use of separate truncated cable portions, e.g., 212 in FIG. 2.

The method has been described above mostly with reference to passing the surgical instrument around the injured bone 20 once to form a single loop. It is to be understood that the method of the invention further comprises passing the surgical instrument 10 around the injured bone 20 a plurality of times, as well as coiling the surgical instrument around an axial portion of the injured bone(s) 20, as shown in FIG. 8.

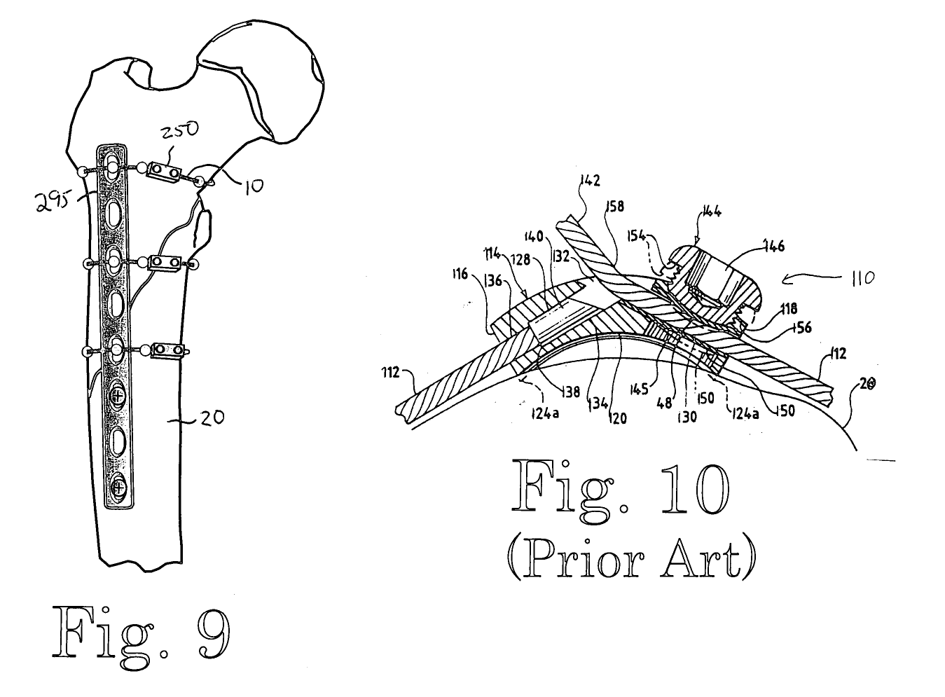

It should be understood that the surgical instrument and methods of this invention, including the above-described embodiments, may be used in conjunction with other surgical devices. For example, the surgical instrument may be used in conjunction with a surgical plate 295 set against an injured bone 20, wherein the surgical instrument 10 passes around the bone 20 and the surgical plate 295 set there against, as shown in FIG. 9. Other devices that may be used in combination with embodiments of the surgical instrument and methods of this invention include, for example, intramedullary metal rods, trochanteric claws or clamps, screw posts, and others.

Advantageously, the surgical instrument and related methods of the present invention permit application of a constant tension to an injured bone, while at the same time providing gaps between bone-contacting parts (enlargements) to permit vascular circulation past the surgical instrument. Additionally, the surgical instrument and related methods of embodiments of the present invention are compatible with conventional clamping and tensioning devices. For example, FIG. 8 illustrates an embodiment of the surgical instrument used in conjunction with a hip prosthesis 290 inserted into the femur with a fracture 20.

The foregoing detailed description of the certain preferred embodiments of the invention has been provided for the purpose of explaining the principles of the invention and its practical application, thereby enabling others skilled in the art to understand the invention for various embodiments and with various modifications as are suited to the particular use contemplated. This description is not intended to be exhaustive or to limit the invention to the precise embodiments disclosed. Modifications and equivalents will be apparent to practitioners skilled in this art and are encompassed within the spirit and scope of the appended claims.

1. A surgical instrument for stabilizing and facilitating recovery of injured bone within a living body, comprising:
a flexible cable having a first end, a second end, and a length between the first and second ends sufficient to wrap around the injured bone; and
a plurality of permanent bone-contacting enlargements fixedly attached to the flexible cable between the first and second ends, the bone-contacting enlargements being spaced apart from one another to provide linking cable portions alternating with the spaced bone-contacting enlargements.

2. A surgical instrument according to claim 1, wherein the flexible cable is formed of a metal.

3. A surgical instrument according to claim 1, wherein the flexible cable is formed of a metal selected from stainless steel and cobalt chrome.

4. A surgical instrument according to claim 1, wherein the flexible cable is axially inelastic.

5. A surgical instrument according to claim 1, wherein the bone-contacting enlargements are obtuse.

6. A surgical instrument according to claim 1, wherein the bone-contacting enlargements comprise beads.

7. A surgical instrument according to claim 1, wherein the bone-contacting enlargements comprise a high molecular weight polymer.

8. A surgical instrument according to claim 1, wherein the bone-contacting obtuse enlargements comprise polyethylene.

9. A surgical instrument according to claim 1, wherein the bone-contacting enlargements comprise a metal.

10. A surgical instrument according to claim 1, wherein the bone-contacting enlargements have peripheries circumferentially surrounding the flexible cable.

11. A surgical instrument according to claim 1, wherein the bone-contacting enlargements each have a respective axial length smaller in dimension than respective axial lengths of adjacent ones of the linking cable portions.

12. A surgical instrument according to claim 1, wherein the flexible cable has an end portion free of the bone-contacting enlargements, the end portion being sufficient in length to permit engagement with a tensioning device.

13. A method for stabilizing and facilitating recovery of injured bone within a living body, said method comprising:
providing a surgical instrument comprising a flexible cable and a plurality of permanent bone-contacting enlargements, the flexible cable having a first end, a second end, and a length sufficient to wrap around the injured bone, the bone-contacting enlargements being fixedly attached to the flexible cable between the first and second ends and being spaced apart from one another to provide linking cable portions alternating with the spaced bone-contacting enlargements;
passing the surgical instrument about the injured bone to contact the bone-contacting enlargements and the injured bone with one another, the bone-contacting enlargements positioning the linking cable portions in spaced relationship to the injured bone;
tensioning the flexible cable about a constricted region of the injured bone while the bone-contacting enlargements retain the linking cable portions in spaced relationship to the injured bone for permitting vascular communication across the constricted region of the injured bone; and
securing surgical instrument about the injured bone.

14. A method according to claim 13, wherein the flexible cable is formed of a metal.

15. A method according to claim 13, wherein the flexible cable is formed of a metal selected from stainless steel and cobalt chrome.

16. A method according to claim 13, wherein the flexible cable is axially inelastic.

17. A method according to claim 13, wherein the bone-contacting enlargements are obtuse.

18. A method according to claim 13, wherein the bone-contacting enlargements comprise beads.

19. A method according to claim 13, wherein the bone-contacting enlargements comprise a high molecular weight polymer.

20. A method according to claim 13, wherein the bone-contacting obtuse enlargements comprise polyethylene.

21. A method according to claim 13, wherein the bone-contacting enlargements comprise a metal.

22. A method according to claim 13, wherein the bone-contacting enlargements have peripheries circumferentially surrounding the flexible cable.

23. A method according to claim 13, wherein the bone-contacting enlargements each have a respective axial length smaller in dimension than respective axial lengths of adjacent ones of the linking cable portions.
